(19) United States
(12) Patent Application Publication
(10) Pub. No.: US 2013/0210932 A1
(43) Pub. Date: Aug. 15, 2013

(54) NOVEL CATIONIC THICKENERS USABLE OVER A WIDE PH RANGE, METHOD FOR THE PREPARATION THEREOF, AND COMPOSITION CONTAINING SAME

(71) Applicants: Olivier Braun, Castres (FR); Paul Mallo, Croissy-Sur-Seine (FR)

(72) Inventors: Olivier Braun, Castres (FR); Paul Mallo, Croissy-Sur-Seine (FR)

(73) Assignee: Societe D'Exploitation De Products Pour Les Industries Chimiques SEPPIC, Paris (FR)

(21) Appl. No.: 13/878,602

(22) PCT Filed: Nov. 28, 2011
(86) PCT No.: PCT/FR2011/052788

§ 371 Date: Apr. 10, 2013

(30) Foreign Application Priority Data
Dec. 2, 2010 (FR) 1060039

(51) Int. Cl. A61K 8/81; A61K 47/32

(52) U.S. Cl. CPC A61K 8/8158; A61K 47/32
USPC 514/772.4

(57) ABSTRACT

Positive latex including an oil phase, an aqueous phase, at least one water-in-oil emulsifying system, and a linear, branched or cross-linked cationic polyelectrolyte resulting from the polymerization, for 100 mol %, of: a) a molar ratio >40% and ≦99% of monomer units originating from at least one cationic monomer; b) a molar ratio ≧1% and <60% of monomer units originating from N-(2-hydroxyethyl)acrylamide; c) optionally, a molar ratio >0% and <30% of monomer units originating from at least one neutral monomer other than the N-(2-hydroxyethyl)acrylamide, on the condition that the molar ratio is strictly less than that of the monomer units originating from N-(2-hydroxyethyl)acrylamide; and d) optionally, a molar ratio >0% and ≦1% of a diethylene or polyethylene cross-linking monomer. A method for preparing and using the positive latex as a thickener for cosmetic or pharmaceutical compositions is also described.

The subject of the invention is novel polymeric cationic thickeners, the process for preparing same and also the use thereof as a thickener and/or emulsifier.

The thickening of aqueous phases is generally carried out by incorporating therein hydrophilic polymers of all types, whether they are synthetic or of natural origin. Among the polymers of natural origin, xanthan or guar gums are quite widely used. However, they have the conventional drawbacks of natural products, namely fluctuating quality and price.

Among the hydrophilic synthetic thickeners most widely used are polymers in the form of powders or of self-invertible inverse latexes. They are used in a wide pH range and are often well tolerated by human beings. Such compositions are described, for example, in the United States patents published under numbers U.S. Pat. No. 5,004,598, U.S. Pat. No. 6,197,287, U.S. Pat. No. 6,136,305 or U.S. Pat. No. 6,346,239 or in the European patent application published under number EP 0 503 853.

These polymers are anionic and are therefore essentially intended for thickening aqueous phases containing the various conventional constituents that can be found in topical formulations of the cosmetic, dermopharmaceutical or pharmaceutical industry. Mention will in particular be made of oils, surfactants (nonionic or anionic) also called emulsifiers, mineral salts and weak acids.

Certain formulations in particular intended for hair care contain cationic surfactants and/or cationic conditioning polymers. In this particular case, the thickeners made up of anionic polymers are not recommended because of the electrostatic interactions between the positive and negative charges which cause precipitation of the polymer, and cationic thickening polymers such as those described in the United States patents published under numbers U.S. Pat. No. 4,806,345 and U.S. Pat. No. 5,100,660 are preferably used.

Although the latter behave satisfactorily in an acidic medium and they are compatible with cationic surfactants, they nevertheless lose their thickening capacity in formulations of which the pH is greater than 7.5 and for which a decrease in the viscosity of the aqueous phase thereof is observed after a few weeks.

Consequently, the inventors have sought to develop electrolyte-resistant thickening polymers of cationic type which operate at alkaline pHs ranging up to 10, i.e. which make it possible to obtain formulae, the viscosities of which remain stable for several months.

According to a first aspect, a subject of the invention is an inverse latex comprising an oil phase, an aqueous phase, at least one water-in-oil emulsifying system, and a linear, branched or crosslinked cationic polyelectrolyte resulting from the polymerization, for 100 mol %:
a) of a molar proportion greater than 40% and less than or equal to 99% of monomeric units resulting from at least one cationic monomer,
b) of a molar proportion greater than or equal to 1% and less than 60% of monomeric units resulting from N-(2-hydroxyethyl)acrylamide, and
c) optionally of a molar proportion greater than 0% and less than 30% of monomeric units resulting from at least one neutral monomer different than said N-(2-hydroxyethyl)acrylamide, it being understood that this molar proportion is strictly less than that of the monomeric units resulting from N-(2-hydroxyethyl)acrylamide, and
d) optionally of a molar proportion greater than 0% and less than or equal to 1% of a diethylenic or polyethylenic crosslinking monomer.

In the context of the present invention, the term “inverse latex” denotes a water-in-oil emulsion of the polyelectrolyte as defined above.

The term “branched polyelectrolyte” denotes a nonlinear polyelectrolyte which has pendant chains so as to obtain, when it is dissolved in water, a highly entangled state resulting in very high viscosities at low rate gradient.

The term “crosslinked polyelectrolyte” denotes a nonlinear polyelectrolyte which is in the form of a three-dimensional network that is water-insoluble but water-swellable and therefore results in the obtaining of a chemical gel.

In the inverse latex as defined above, the “water-in-oil” (W/O) emulsifying system consists either of a single surfactant or of a mixture of surfactants, on the condition that said surfactant or said mixture has an HLB value sufficiently low to induce a water-in-oil emulsion. Mention is made, for example, of sorbitan esters, for instance sorbitan oleate, sold by the company SEPPIC under the name Montane™ 80, sorbitan isostearate, sold by the company SEPPIC under the name Montane™ 70 or sorbitan sesquioleate sold by the company SEPPIC under the name Montane™ 83. Mention is also made of certain polyethoxylated sorbitan esters, for example the pentaethoxylated sorbitan monooleate sold by the company SEPPIC under the name Montanox™ 81 or the pentaethoxylated sorbitan isostearate sold under the name Montanox™ 71 by the company SEPPIC. Mention is also made of polyesters having a molecular weight between 1000 and 3000, produced from condensation between a poly(isobutenyl)succinic acid or its anhydride and such as Hypermer™ 2296 sold by the company Uniqema or, finally, block copolymers having a molecular weight between 2500 and 3500, for instance Hypermer™ B246 sold by the company Uniqema or Simaline™ IE 200 sold by the company SEPPIC.

The inverse latex as previously defined generally comprises between 0.5% by weight and 10% by weight of said “water-in-oil” emulsifying system.

The inverse latex generally contains between 1% and 50% by weight of water.

The oil phase of the self-invertible inverse latex described above is made up:
either of a mineral oil, or of a mixture of mineral oils, containing saturated hydrocarbons of paraffin, isoparaffin or cycloparaffin type, having, at ambient temperature, a density between 0.7 and 0.9 and a boiling point above 180° C., such as, for example, Isopar™ M or Isopar™ L, Exxol™ D 100 S sold by Exxon or the mineral white oils in accordance with the FDA 21 CFR 172.878 and FR 178.3620(a) regulations, such as Marcol™ 52 or Marcol™ 82, also sold by Exxon;
or of a synthetic oil, or of a synthetic oil mixture, such as hydrogenated polyisobutenes, in particular those sold in France by the company Ets B. Hossow and Cie under the name Parleam-Polysynlane™ and cited in Michel and Irene Ash;

Thesaurus of Chemical products, Chemise Publicite Cos, Ince. 1986 Volume I, page 211 (ISBN 0 7131 36030); polydecenes; the isohexadecane identified in Chemical Abstracts by the number RN=93685-80-4 and which is a mixture of C₁₂, C₁₆ and C₂₀ isoparaffins containing at least 97% of C₁₆ isoparaffins, among which the main constituent is 2,2,4,4,6,8,8-heptamethylnonane (RN=4390-04-9), sold in France by the company Bayer; isododecane, sold in France by the company Bayer;
or of a vegetable oil, or of a mixture of vegetable oils, such as squalane which is identical in Chemical Abstracts by the number RN=111-01-3 and which is a mixture of hydrocarbons containing more than 80% by weight of 2,6,10,15,19,23-hexamethyltetracosane, or a vegetable oil of ester or triglyceride type, for instance coco-caprylate/caprate, for example DUB™ 810C provided by the company Dubois, or else jojoba oil;
or of a mixture of several of these various oils.

The inverse latex as previously defined generally comprises, for 100% by weight, from 5% to 50% by weight of oil.

The term “cationic monomer” principally denotes an aliphatic monomer comprising a quaternary ammonium function and at least one unsaturated carbon-carbon bond. Such a monomer is generally available in the form in particular of salts.

The term “salts” denotes more particularly halides, such as bromides, chlorides or iodides, of said monomers comprising a quaternary ammonium function.

According to one particular aspect, a subject of the invention is an inverse latex as previously defined, for which the monomeric units resulting from at least one cationic monomer, which are constituents of said cationic polyelectrolyte, result from the following quaternary ammonium salts;
N,N,N-trimethyl-3-[(2-methyl-1-oxo-2-propenyl)amino]-propanammonium salts,
N,N,N-trimethyl-3-[(1-oxo-2-propenyl)amino]propan-ammonium salts; or
diallyldimethylammonium salts, and more particularly:
N,N,N-trimethyl-3-[(2-methyl-1-oxo-2-propenyl)amino]-propanammonium chloride;
N,N,N-trimethyl-3-[(1-oxo-2-propenyl)amino]propan-ammonium chloride; or
diallyldimethylammonium chloride.

The expression “neutral monomer different than said N-(2-hydroxyethyl)acrylamide” denotes monomers comprising no strong or weak acid function nor any positively charged group. They are more particularly chosen from vinylpyrrolidone, diacetoneacrylamide, N,N-dimethylacrylamide, or N-[2-hydroxy-1,1-bis-(hydroxymethyl)ethyl]propenamide [or tris(hydroxy-methyl)acrylamidomethane or N-[tris(hydroxy-methyl)methyl]acrylamide, also known as THAM].

According to one particular aspect of the present invention, the cationic polyelectrolyte as defined above is characterized in that the molar proportion of monomeric units resulting from the cationic monomer(s) is less than or equal to 95% and more particularly less than or equal to 90%.

According to another particular aspect, the cationic polyelectrolyte as defined above is characterized in that the molar proportion of monomeric units resulting from the cationic monomer(s) is greater than or equal to 50%, more particularly greater than or equal to 60% and even more particularly greater than or equal to 70%.

More particularly, a subject of the invention is an inverse latex as previously defined, for which the molar proportion of monomeric units resulting from the cationic monomer(s) is greater than or equal to 50% and less than or equal to 95%.

According to another particular aspect, a subject of the invention is an inverse latex as previously defined, for which said monomeric units resulting from at least one cationic monomer, which are constituents of said cationic polyelectrolyte, result solely from an N,N,N-trimethyl-3-[(1-oxo-2-propenyl)amino]propanammonium salt and in particular from N,N,N-trimethyl-3-[(1-oxo-2-propenyl)amino]propan-ammonium chloride (APTAC™).

According to another particular aspect of the present invention, the polyelectrolyte as defined above is characterized in that the molar proportion of monomeric units resulting from N-(2-hydroxyethyl)acrylamide is greater than or equal to 5% and more particularly greater than or equal to 10%.

According to another particular aspect, the cationic polyelectrolyte as defined above is characterized in that the molar proportion of monomeric units resulting from N-(2-hydroxyethyl)acrylamide is less than or equal to 50%, more particularly less than or equal to 40% and even more particularly less than or equal to 30%.

More particularly, a subject of the invention is an inverse latex as previously defined, for which the molar proportion of monomeric units resulting from N-(2-hydroxyethyl)acrylamide is greater than or equal to 5% and less than than or equal to 50%.

A subject of the present invention is more particularly an inverse latex as previously defined, in which the linear, branched or crosslinked cationic polyelectrolyte results from the polymerization, for 100 mol %:
a) of a molar proportion greater than or equal to 60% and less than or equal to 90% of monomeric units resulting from at least one cationic monomer,
b) of a molar proportion greater than or equal to 10% and less than or equal to 40% of monomeric units resulting from N-(2-hydroxyethyl)acrylamide, and
c) optionally of a molar proportion greater than 0% and less than or equal to 20% of monomeric units resulting from at least one neutral monomer different than said N-(2-hydroxyethyl)acrylamide, it being understood that this molar proportion is strictly less than that of the monomeric units resulting from N-(2-hydroxyethyl)acrylamide, and
d) optionally of a molar proportion greater than or equal to 0.005% and less than or equal to 1% of a diethylenic or polyethylenic crosslinking monomer.

According to another particular aspect of the present invention, a subject thereof is an inverse latex as previously defined, in which said cationic polyelectrolyte is crosslinked.

In the latter case, a subject of the invention is more particularly an inverse latex as defined above, for which said diethylenic or polyethylenic or polyelectrolyte crosslinking monomer is chosen from diallyloxyacetic acid or the sodium or potassium salt thereof, triallylamine, trimethylolpropane triacrylate, ethylene glycol dimethacrylate, diethylene glycol diacrylate, diallylurea or methylenebis(acrylamide), and most particularly, said diethylenic or polyethylenic or polyelectrolyte crosslinking monomer is methylenebis(acrylamide).

According to another particular aspect, said diethylenic or polyethylenic or polyelectrolyte crosslinking monomer is used in the molar proportion, expressed relative to the monomers used, of greater than 0.05%, more particularly from 0.01% to 0.2% and most particularly from 0.01% to 0.1%.

According to another most particular aspect, a subject of the invention is an inverse latex as previously defined, in which the crosslinked cationic polyelectrolyte results from the polymerization, for 100 mol %:
a) of a molar proportion greater than or equal to 70% and less than or equal to 90% of monomeric units resulting from N,N,N-trimethyl-3-[(1-oxo-2-propenyl)amino]propanammonium chloride,
b) of a molar proportion greater than or equal to 10% and less than or equal to 30% of monomeric units resulting from N-(2-hydroxyethyl)acrylamide, and
d) of a molar proportion greater than or equal to 0.01% and less than or equal to 0.2% of methylenebis(acrylamide).

According to another aspect of the present invention, the inverse latex as previously defined is characterized in that it also comprises an “oil-in-water” emulsifying system.

Said “oil-in-water (0/W) emulsifying system” consists either of a single surfactant or of a mixture of surfactants, on the condition that said surfactant or said mixture has an HLB value sufficiently high to induce an oil-in-water emulsion. Mention is made, for example, of:
ethoxylated sorbitan esters, for instance sorbitan oleate polyethoxylated with 20 mol of ethylene oxide, sold by the company SEPPIC under the name Montanox™ 80 or sorbitan laurate polyethoxylated with 20 mol of ethylene oxide, sold by the company SEPPIC under the name Montanox™ 20:
the castor oil polyethoxylated with 40 mol of ethylene oxide sold under the name Simulsol™ OL50;
the decaethoxylated oleodecyl alcohol sold by the company SEPPIC under the name Simulsol™ OC 710;
the heptaethoxylated lauryl alcohol sold under the name Simulsol™ P7; or
the polyethoxylated sorbitan hexaoleates sold by the company SEPPIC under the name Simaline IE 400.

When it also comprises an “oil-in-water” emulsifying system, the inverse latex as previously described is then self-invertible; in this case, it generally comprises from 1% by weight to 15% by weight of said “oil-in-water” emulsifying system.

The inverse latex according to the invention can also contain various additives, such as complexing agents or chain limiters.

According to another particular aspect of the present invention, a subject thereof is an inverse latex as previously defined, characterized in that it comprises, for 100% by weight, from 15% to 60% by weight, and preferably from 25% to 40% by weight, of said cationic polyelectrolyte.

According to another particular aspect of the present invention, a subject thereof is an inverse latex as previously defined, characterized in that it comprises, for 100% by weight, more than 60% up to 80% by weight, and preferably more than 60% to 70% by weight, of said cationic polyelectrolyte.

According to another aspect of the present invention, a subject thereof is a process for preparing the inverse latex as previously defined, comprising the following steps:

A step a) during which an aqueous solution comprising the monomers and the optional additives which are hydrophilic is emulsified in an oil phase comprising the monomers and the optional additives which are lipophilic in the presence of said water-in-oil emulsifying system;

A step b) during which the polymerization reaction is initiated by introducing into the emulsion formed at the end of step a) a free-radical initiator and optionally a coinitiator, and then left to take place so as to obtain said inverse latex.

During step b) of the process as defined, the polymerization reaction is generally initiated by an oxidation/reduction couple which generates hydrogen sulfite (HSO₃) ions, such as the cumene hydroperoxide/sodium metabisulfite (Na₂S₂O₅) couple or the tert-butyl hydroperoxide/sodium metabifulsite couple, at a temperature of less than or equal to 10° C., if desired accompanied by a polymerization coinitiator, for instance azobis(isobutyronitrile), dilauryl peroxide or sodium persulfate, and then carried out either quasiadiabatically up to a temperature greater than or equal to 50° C., or by controlling the temperature.

According to one particular aspect, the process as defined above also comprises a step c) during which said oil-in-water emulsifying system is added to the inverse latex formed at the end of step b), so as to obtain a self-invertible inverse latex.

During step c) of the process as defined above, the addition of said oil-in-water emulsifying system is generally carried out at a temperature of less than or equal to 50° C.

According to another particular aspect, the process as defined above also comprises a step b₁ during which the inverse latex resulting from step b) is concentrated so as to obtain a concentrated inverse latex, before the implementation, where appropriate, of step c).

According to another particular aspect, the process as defined above also comprises a step c₁ during which the self-invertible inverse latex resulting from step c) is concentrated, so as to obtain said concentrated self-invertible inverse latex.

During step b₁ or step c₁ of the process as defined above, the concentrating of the medium is generally carried out by distillation until the desired content of cationic polyelectrolyte within the inverse latex which is the subject of the present invention is achieved.

According to one particular aspect, the process as defined above also comprises a step d) during which the inverse latex resulting from step b), the concentrated inverse latex resulting from step b1), the self-invertible inverse latex resulting from step c) or the concentrated self-invertible inverse latex resulting from step c1) is spray-dried, so as to form a powder of said cationic polyelectrolyte.

A subject of the invention is also a powder of the linear, branched or crosslinked cationic polyelectrolyte, characterized in that it is obtained by means of the process as defined above.

By virtue of its cationic nature, the polyelectrolyte which is the subject of the present invention and also the inverse latexes and the self-invertible inverse latexes comprising same are advantageously used as thickeners and/or as emulsifiers in cosmetic or pharmaceutical compositions intended for hair care and/or hair conditioning.

Consequently, according to another aspect, a subject of the invention is the use of the inverse latex as previously defined or of the powder obtained by means of the process as previously defined, as a thickener and/or as an emulsifier for cosmetic or pharmaceutical compositions and more particularly those intended for hair care and/or hair conditioning.

The powder or the optionally self-invertible inverse latex, which are subjects of the present invention, can be formulated in cosmetic or pharmaceutical formulae such as mousses, gels, lotions, sprays, shampoos, conditioners, hand and body lotions, and sunscreens, and more particularly in care products.

In the case of hair treatment or upkeep, such cosmetic or pharmaceutical compositions are usually in the form of shampoos, of emulsions, of microemulsions and, in particular in the case of conditioners, of vaporizable emulsions.

According to a final aspect, a subject of the invention is a cosmetic or pharmaceutical composition characterized in that it contains, as emulsifier and/or thickener, an effective amount of inverse latex as previously defined or of the powder obtained by means of the process as previously defined.

The term “effective amount” is intended to mean a weight proportion of between approximately 1% and approximately 10% by weight of the inverse latex as previously defined and approximately 0.2% by weight to approximately 5% of the powder as previously defined.

The following examples illustrate the invention without, however, limiting it.

### A]—Examples of Preparation of Inverse Latexes or of Powders According to the Invention

### EXAMPLE 1

### Preparation of a Powder (Powder P1)

### Preparation of the Powder

a) An aqueous phase is prepared by successively mixing:
   25.3 g of N-(2-hydroxyethyl)acrylamide;
   242.4 g of a commercial solution containing 75% of N,N,N-trimethyl-3-[(1-oxo-2-propenyl)amino]propan-ammonium chloride (APTAC);
   0.066 g of methylenebis(acrylamide);
   0.27 g of a commercial solution containing 40% of the sodium salt of triaminepentaacetic acid;
   approximately 2.1 g of a normal aqueous solution of hydrochloric acid, so as to bring the pH to 5; and
   deionized water so as to bring the total weight of the aqueous phase to 325.5 g.
b) An oil phase is prepared by successively mixing:
   129.6 g of Isopar™ M (C13-C14 isoparaffin);
   12.5 g of Montane™ 70 (sorbitan isostearate);
   0.04 g of azobis(isobutyronitrile).
c) The two phases are then intimately mixed by means of an Ultra Turrax™ turbine so as to form a water-in-oil emulsion.
d) The emulsion obtained is cooled to approximately 10° C. and placed under nitrogen sparging for approximately 60 minutes. The polymerization is then initiated by incorporating therein an oxidation/reduction couple consisting of:
   8 g of a constituted cumene hydroperoxide solution (0.043 g in 20 ml of water), and
   20 g of an aqueous solution containing 0.042 g of sodium metabisulfite.
e) At the end of the reaction, after a conventional step devoted to destroying the residual monomers, an inverse latex is obtained which is spray-dried. The expected cationic polyelectrolyte powder comprising 10% by weight of water is thus obtained.

### Viscosimetric Analysis of the Powder

Viscosity of an aqueous dispersion comprising 1.5% by weight of the powder obtained: 139 000 mPa·s (Brookfield RVT, spindle 6, rotational speed 5). Viscosity of an aqueous dispersion comprising 1.5% by weight of the powder obtained and 0.1% by weight of sodium chloride: 15 540 mPa·s (Brookfield RVT, spindle 3, rotational speed 5).

It is noted that this value is identical at pH 6.8 or at pH 10 and that it remains constant after six months of storage at ambient temperature.

### EXAMPLE 2

### Preparation of a Self-Invertible Inverse Latex According to the Invention

### (Inverse Latex L1)

### Preparation of the Self-Invertible Inverse Latex

a) An aqueous phase is prepared by successively mixing:
   50.6 g of N-(2-hydroxyethyl)acrylamide;
   485 g of a commercial solution containing 75% of N,N,N-trimethyl-3-[(1-oxo-2-propenyl)amino]propan-ammonium chloride (APTAC);
   0.13 g of methylenebis(acrylamide);
   0.45 g of a commercial solution containing 40% of the sodium salt of triaminepentaacetic acid;
   approximately 4 g of a 1N normal aqueous solution of hydrochloric acid so as to bring the pH to 5; and
   deionized water so as to bring the total weight of the aqueous phase to 651 g.
b) An oil phase is prepared by successively mixing:
   259 g of Isopar™ M
   25 g of Montane™ 70 (sorbitan isostearate);
   0.08 g of azobis(isobutyronitrile).
c) The two phases are then intimately mixed by means of an Ultra Turrax™ turbine so as to form a water-in-oil emulsion.
d) The whole mixture is then cooled to approximately 10° C. and placed under nitrogen sparging for approximately 60 minutes. The polymerization is then initiated by incorporating therein an oxidation/reduction couple consisting of:
   8 g of a constituted cumene hydroperoxide solution (0.043 g in 20 cm³ of water), and
   20 g of an aqueous solution containing 0.042 g of sodium metabisulfite.
e) At the end of the reaction, after a conventional step devoted to destroying the residual monomers, an inverse latex is obtained. 50 g of Simulsol™ P7 (heptaethoxylated lauryl alcohol) is then added thereto with stirring at 35° C. and a self-invertible inverse latex (referred to in the following examples as inverse latex L1) is obtained.

### Viscosimetric Analysis of the Self-Invertible Inverse Latex

Viscosity at 3% in water at pH =6 of the self-invertible inverse latex:
122 600 mPa·s (Brookfield RVT, spindle 6; rotational speed 5).

Viscosity at 3% in water containing 0.1% of sodium chloride, of the self-invertible inverse latex: 5 740 mPa·s (Brookfield RVT, spindle 3, rotational speed 5).

It is noted that, contrary to what happens for the self-invertible inverse latexes which are equivalent but have no monomeric units resulting from N-(2-hydroxyethyl)acrylamide, this value is identical at pH=6, pH=8 or at pH=10 and that it remains constant after six months of storage at ambient temperature.

### B]—Formulation Examples

### EXAMPLE 3

### Antistress Hair Care Product

Formula

| Phase A | |
|---|---|
| Water: | q.s. 100% |
| Xanthan gum | 0.50% |
| Phase B | |
| Sepicap ™ MP: | 3.00% |
| Phase C | |
| Inverse latex L1 | 4.00% |
| Phase D | |
| Butylene glycol: | 5.00% |
| Lanol ™ 99: | 5.00% |
| Sepicide ™ HB: | 0.30% |
| Sepicide ™ Cl: | 0.20% |
| Fragrance | 0.20% |

### Procedure

The xanthan gum is dispersed in the water with a deflocculator. Sepicap™ MP is then added, followed by the inverse latex of example 1; it is dispersed and then the ingredients of phase D are added.

### EXAMPLE 4

### Restructuring Cream Mask for Stressed and Embrittled Hair

Formula

| Phase A | |
|---|---|
| Montanov ™ 82: | 3.00% |
| Lanol ™ P: | 6.00% |
| Amonyl ™ DM: | 1.00% |
| Isostearyl isononanoate: | 5.00% |
| Powder P1: | 2.50% |
| Phase B | |
| Water: | q.s. 100% |
| Phase C | |
| Sepicide ™ MP: | 3.00% |
| Sepicide ™ HB: | 0.30% |
| Sepicide ™ Cl: | 0.20% |

### Procedure

Phase A is melted at 75° C. Phase B is heated at 75° C. A is emulsified in B. At around 40° C., the constituents of phase C are introduced.

### EXAMPLE 5

### Purifying Facial Gel

Formula

| | |
|---|---|
| Phase A | |
| Montaline ™ C 40: | 7.00% |
| Pearlescent base 2078: | 5.00% |
| Inverse latex L1: | 2.00% |
| Phase B | |
| Water: | q.s. 100% |

### EXAMPLE 6

### Coloring Shampoo

Formula

| | |
|---|---|
| Phase A | |
| Montaline ™ C 40: | 15.00% |
| Disodium cocoamphoacetate: | 5.00% |
| Cetrimonium chloride: | 1.00% |
| Sepiperl ™ N: | 3.00% |
| Inverse latex 1: | 3.00% |
| Phase B | |
| Color | q.s. |
| Water | q.s. 100% |

### EXAMPLE 7

### Fluid Emulsion at Alkaline pH

| | |
|---|---|
| Marcol ™ 82: | 5.0% |
| Sodium hydroxide: | 10.0% |
| Water: | q.s. 100% |
| Powder P1: | 1.5% |

### EXAMPLE 8

### Restructuring Rinse-Off Cream Mask for Stressed and Embrittled Hair

| | |
|---|---|
| Ketrol ™ T: | 0.5% |
| Pecosil ™ SPP50: | 0.75% |
| N-Cocoyl amino acids: | 0.70% |
| Butylene glycol: | 3.0% |
| Inverse latex L1: | 3.0% |
| Montanov ™ 82: | 3.0% |
| Jojoba oil: | 1.0% |
| Lanol ™ P: | 6.0% |
| Amonyl ™ DM: | 1.0% |
| Lanol ™ 99: | 5.0% |
| Sepicide ™ HB: | 0.3% |
| Sepicide ™ Cl: | 0.2% |
| Fragrance: | 0.2% |
| Water: | q.s. 100% |

### EXAMPLE 9

### Hair Lotion

| | |
|---|---|
| Butylene glycol: | 3.0% |
| Inverse latex 1: | 3.0% |
| Simulsol ™ 1293: | 3.0% |
| Lactic acid: | q.s. pH = 6 |
| Sepicide ™ HB: | 0.2% |
| Sepicide ™ Cl: | 0.3% |
| Fragrance: | 0.3% |
| Water: | q.s. 100% |

### EXAMPLE 10

### Protecting and Relaxing Shampoo

| | |
|---|---|
| Amonyl ™ 675 SB: | 5.0% |
| Sodium lauroyl ether sulfate at 28%: | 35.0% |
| Powder P1: | 3.0% |
| Sepicide ™ HB: | 0.5% |
| Sepicide ™ Cl: | 0.3% |
| Sodium hydroxide: | q.s. pH = 7.2 |
| Fragrance: | 0.3% |
| Dye (FDC blue 1/yellow 5): | q.s. |
| Water: | q.s. 100% |

### EXAMPLE 11

### Leave-On Protector; Antistress Hair Care Product

| | |
|---|---|
| Ketrol ™ T: | 0.5% |
| Mixture of cocoyl amino acids: | 3.0% |
| Butylene glycol: | 5.0% |
| DC 1501: | 5.0% |
| Inverse latex L1: | 4.0% |
| Sepicide ™ HB: | 0.5% |
| Sepicide ™ Cl: | 0.3% |
| Fragrance: | 0.3% |
| Water: | q.s. 100% |

The definitions of the commercial products used in the examples are the following:
Montaline™ C40: (cocamoniumcarbamoyl chloride) sold by SEPPIC.
Sepiperl™ N: (cocoyl glucoside/cocoyl alcohol) sold by SEPPIC.
Amonyl™ DM: (quaternium 82) sold by SEPPIC.
Sepicap™ MP: (sodium cocoyl amino acids/potassium dimethicone copolyol panthenyl phosphate) sold by SEPPIC.
Simulsol™ 1293 is hydrogenated and ethoxylated castor oil, with an ethoxylation number equal to 40, sold by the company SEPPIC.
Ketrol™ T is xanthan gum sold by the company Kelco.
Lanol™ 99 is isononyl isononanoate sold by the company SEPPIC.
DC1501 is a mixture of cyclopentasiloxane and dimethiconol sold by the company Dow Chemical.
Montanov™ 82 is an emulsifier based on cetearyl alcohol and cocoylglucoside.
Sepicide™ Cl, imidazolidine urea, is a preservative sold by the company SEPPIC.
Sepicide™ HB, which is a mixture of phenoxyethanol, methylparaben, ethylparaben, propylparaben and butylparaben, is a preservative sold by the company SEPPIC.
Lanol™ P is an additive with a stabilizing effect, sold by the company SEPPIC.

1. An inverse latex comprising an oil phase, an aqueous phase, at least one water-in-oil emulsifying system, and a linear, branched or crosslinked cationic polyelectrolyte resulting from the polymerization, for 100 mol %:
a) of a molar proportion greater than 40% and less than or equal to 99% of monomeric units resulting from at least one cationic monomer,
b) of a molar proportion greater than or equal to 1% and less than 60% of monomeric units resulting from N-(2-hydroxyethyl) acrylamide, and
c) optionally of a molar proportion greater than 0% and less than 30% of monomeric units resulting from at least one neutral monomer different than said N-(2-hydroxyethyl)acrylamide, it being understood that this molar proportion is strictly less than that of the monomeric units resulting from N-(2-hydroxyethyl) acrylamide, and
d) optionally of a molar proportion greater than 0% and less than or equal to 1% of a diethylenic or polyethylenic crosslinking monomer.

2. The inverse latex as defined in claim 1, for which the monomeric units resulting from at least one cationic monomer, which are constituents of said cationic polyelectrolyte, result from the following quaternary ammonium salts:
N,N,N-trimethyl-3-[(2-methyl-1-oxo-2-propenyl)-amino]propanammonium salts;
N,N,N-trimethyl-3-[(1-oxo-2-propenyl)amino]-propanammonium salts; or
diallyldimethylammonium salts.

3. The inverse latex as defined in claim 1, for which the molar proportion of monomeric units resulting from the cationic monomer(s) is greater than or equal to 50% and less than or equal to 95%.

4. The inverse latex as defined in claim 2, for which said monomeric units resulting from at least one cationic monomer, which are constituents of said cationic polyelectrolyte, result solely from an N,N,N-trimethyl-3-[(1-oxo-2-propenyl)amino]propanammonium salt and in particular from N,N,N-trimethyl-3-[(1-oxo-2-propenyl)amino]propanammonium chloride.

5. The inverse latex as defined in claim 1, for which the molar proportion of monomeric units resulting from N-(2-hydroxyethyl)acrylamide is greater than or equal to 5% and less than or equal to 50%.

6. The inverse latex as defined in claim 1, in which the linear, branched or crosslinked cationic polyelectrolyte results from the polymerization, for 100 mol %:
a) of a molar proportion greater than or equal to 60% and less than or equal to 90% of monomeric units resulting from at least one cationic monomer,
b) of a molar proportion greater than or equal to 10% and less than or equal to 40% of monomeric units resulting from N-(2-hydroxyethyl)acrylamide, and
c) optionally of a molar proportion greater than 0% and less than or equal to 20% of monomeric units resulting from at least one neutral monomer different than said N-(2-hydroxyethyl)acrylamide, it being understood that the molar proportion is strictly less than that of the monomeric units resulting from N-(2-hydroxyethyl) acrylamide, and
d) optionally of a molar proportion greater than or equal to 0.005% and less than or equal to 1% of a diethylenic or polyethylenic crosslinking monomer.

7. The inverse latex as defined in claim 1, in which said cationic polyelectrolyte is crosslinked.

8. The inverse latex as defined in claim 7, for which said diethylenic or polyethylenic or polyelectrolyte crosslinking monomer is chosen from diallyloxyacetic acid or the sodium or potassium salt thereof, triallylamine, trimethylolpropane triacrylate, ethylene glycol dimethacrylate, diethylene glycol diacrylate, diallylurea or methylenebis(acrylamide).

9. The inverse latex as defined in claim 1, in which the crosslinked cationic polyelectrolyte results from the polymerization, for 100 mol %:
a) of a molar proportion greater than or equal to 70% and less than or equal to 90% of monomeric units resulting from N,N,N-trimethyl-3-[(1-oxo-2-propenyl)amino]propanammonium chloride,
b) of a molar proportion greater than or equal to 10% and less than or equal to 30% of monomeric units resulting from N-(2-hydroxyethyl)acrylamide, and
d) of a molar proportion greater than or equal to 0.01% and less than or equal to 0.2% of methylenebis(acrylamide).

10. The inverse latex as defined in claim 1, characterized in that it also comprises an “oil-in-water” emulsifying system.

11. A process for preparing the inverse latex as defined in claim 1, comprising the following steps:
a step a) during which an aqueous solution comprising the monomers and the optional additives which are hydrophilic is emulsified in an oil phase comprising the monomers and the optional additives which are lipophilic in the presence of said water-in-oil emulsifying system;
a step b) during which the polymerization reaction is initiated by introducing into the emulsion formed at the end of step a) a free-radical initiator and optionally a coinitiator, and then left to take place so as to obtain said inverse latex.

12. The process as defined in claim 11, also comprising a step c) during which said oil-in-water emulsifying system is added to the inverse latex formed at the end of step b), so as to obtain a self-invertible inverse latex.

13. The process as defined in claim 11, also comprising a step b₁ during which the inverse latex resulting from step b) is concentrated so as to obtain a concentrated inverse latex, before the implementation, where appropriate, of step c).

14. The process as defined in claim 13, also comprising a step c₁ during which the self-invertible inverse latex resulting from step c) is concentrated, so as to obtain a concentrated self-invertible inverse latex.

15. The process as defined in claim 11, also comprising a step d) during which the inverse latex resulting from step b), the concentrated inverse latex resulting from step b1), the self-invertible inverse latex resulting from step c) or the concentrated self-invertible inverse latex resulting from step c1) is spray-dried, so as to form a powder of said cationic polyelectrolyte.

16. (canceled)

17. A cosmetic or pharmaceutical composition characterized in that it contains, as emulsifier and/or thickener, an effective amount of the inverse latex as defined in claim 1.
